(19) United States
(12) Patent Application Publication
(10) Pub. No.: US 2010/0191291 A1
(43) Pub. Date: Jul. 29, 2010

(54) BONE FIXATION DEVICE AND METHODS FOR TREATING SPINAL STENOSIS

(76) Inventors: Christopher U. PHAN, San Leandro, CA (US); Andrew Kohm, San Mateo, CA (US); Hugues F. Malandain, Mountain View, CA (US)

Correspondence Address: MEDTRONIC;Attn: Noreen Johnson - IP Legal Department, 2600 Sofamor Danek Drive, MEMPHIS, TN, 38132, US

(21) Appl. No.: 12/361,931

(22) Filed: Jan. 29, 2009

(51) Int. Cl. A61B 17/04

(52) U.S. Cl. 606/301; 606/319; 606/305

(57) ABSTRACT

An apparatus includes a screw, an actuator and a spacer. The screw has a distal end portion and a proximal end portion. The distal end portion of the screw is configured to be threaded into a bone tissue. The proximal end portion of the screw has a surface. The actuator is threadedly coupled to the screw, and has an actuation surface defining a line substantially non-parallel to and substantially non-normal to a longitudinal axis of the screw. The spacer has a first surface substantially parallel to and in contact with the surface of the proximal end portion of the screw. The spacer has a second surface substantially parallel to and in contact with the actuation surface of the actuator. The actuator is configured to move the spacer relative to the screw between a first position and a second position.

### BACKGROUND

The invention relates generally to medical devices and procedures. More particularly, the invention relates to adjustable bone screws and methods for treating spinal stenosis.

Spinal stenosis is a progressive narrowing of the spinal canal that causes compression of the spinal cord and nerve roots extending from the spinal cord. Each vertebra in the spinal column has an opening extending therethrough. The openings of the vertebrae are aligned vertically to form the spinal canal, within which the spinal cord is disposed. As the spinal canal narrows from spinal stenosis, the spinal cord and nerve roots extending from the spinal cord and between adjacent vertebrae are compressed and may become inflamed. Spinal stenosis can cause pain, weakness, numbness, burning sensations, tingling, and in particularly severe cases, may cause loss of bladder or bowel function, or paralysis.

Mild cases of spinal stenosis may be treated with rest or restricted activity, non-steroidal anti-inflammatory drugs (e.g., aspirin), corticosteroid injections (epidural steroids), and/or physical therapy. In certain instances, the compression of the nerve roots may be surgically corrected (e.g., via a decompressive laminectomy) as the patient has increasing pain. In some known surgical procedures, bone and other tissue that has impinged upon the spinal canal and/or exerted pressure on the spinal cord can be removed. In other known surgical procedures, two adjacent vertebrae may be fused to prevent an area of instability, improper alignment or slippage, such as that caused by spondylolisthesis. In yet other known surgical procedures, spacers and/or surgical cables can be disposed between and/or about adjacent spinous processes to limit the movement between adjacent vertebrae.

Such known procedures, however, are not well suited to treat spinal stenosis in the L5-S1 location of the spinal column because the sacrum does not include a spinous process having sufficient area to support implants, tethers or the like. Moreover, known procedures for treating spinal stenosis in the L5-S1 location of the spinal column often employ inserting multiple tools through one or more incisions to perform the desired operations.

Thus, a need exists for improved bone fixation devices and methods for treating spinal stenosis. More particularly, a need exists for methods for treating spinal stenosis in the L5-S1 location.

### SUMMARY

Apparatus and methods for treating spinal stenosis are described herein. In some embodiments, an apparatus includes a screw, an actuator and a spacer. The screw has a distal end portion and a proximal end portion. The distal end portion of the screw is configured to be threaded into a bone tissue. The proximal end portion of the screw has a surface. The actuator is threadedly coupled to the screw, and has an actuation surface defining a line substantially non-parallel to and substantially non-normal to a longitudinal axis of the screw. The spacer has a first surface substantially parallel to and in contact with the surface of the proximal end portion of the screw. The spacer has a second surface substantially parallel to and in contact with the actuation surface of the actuator. The actuator is configured to move the spacer relative to the screw between a first position and a second position.

### BRIEF DESCRIPTION OF THE DRAWINGS

FIGS. 1 and 2 are cross-sectional schematic illustrations of a bone fixation device according to an embodiment, in a first configuration and a second configuration, respectively.

FIGS. 3 and 4 are schematic illustrations of a bone fixation device according to an embodiment, in a first configuration and a second configuration, respectively.

FIG. 5 is a perspective view of a bone fixation device according to an embodiment, in a first configuration.

FIG. 6 is a top view of the bone fixation device shown in FIG. 5, in the first configuration.

FIG. 7 is a cross-sectional view of the bone fixation device shown in FIGS. 5 and 6 taken along line X-X in FIG. 6, in the first configuration.

FIG. 8 is a perspective view of the bone fixation device shown in FIG. 5, in a second configuration.

FIG. 9 is a top view of the bone fixation device shown in FIG. 8, in the second configuration.

FIG. 10 is a cross-sectional view of the bone fixation device shown in FIGS. 8 and 9 taken along line X-X in FIG. 9, in the second configuration.

FIG. 11 is a perspective view of a portion of the bone fixation device shown in FIG. 5.

FIG. 12 is a perspective view of a spacer of the bone fixation device shown in FIG. 5.

FIG. 13 is a perspective view of an actuator of the bone fixation device shown in FIG. 5.

FIG. 14 is a perspective view an insertion tool according to an embodiment coupled to the bone fixation device shown in FIG. 10.

FIGS. 15 and 16 are cross-sectional views of a portion of the insertion tool shown in FIG. 14 in a first configuration and a second configuration, respectively.

FIGS. 17 and 18 are perspective views of a bone fixation device according to an embodiment.

FIG. 19 is a cross-sectional perspective view of the bone fixation device shown in FIGS. 17 and 18.

FIG. 20 is an exploded perspective view of the bone fixation device shown in FIGS. 17 and 18.

FIG. 21 is a perspective view an actuation tool according to an embodiment coupled to the bone fixation device shown in FIGS. 17 and 18.

FIG. 22 is a flow chart of a method of inserting a bone stabilizer according to an embodiment.

FIGS. 23 and 24 are posterior views of a portion of a spinal column having the bone fixation device shown in FIGS. 17-18 inserted therein according to the method illustrated in FIG. 22.

### DETAILED DESCRIPTION

In some embodiments, an apparatus includes a screw, an actuator and a spacer. The screw has a distal end portion and a proximal end portion. The distal end portion of the screw is configured to be threaded into a bone tissue. The proximal end portion of the screw has a surface. The actuator is threadedly coupled to the screw, and has an actuation surface defining a line substantially non-parallel to and substantially non-normal to a longitudinal axis of the screw. The spacer has a first surface substantially parallel to and in contact with the surface of the proximal end portion of the screw. The spacer has a second surface substantially parallel to and in contact with the actuation surface of the actuator. The actuator is configured to move the spacer relative to the screw between a first position and a second position. In some embodiments, the first surface of the spacer defines a groove, and the surface of the proximal end portion of the screw includes a protrusion configured to be matingly received within the groove. In this manner, the first surface of the spacer is matingly and movably coupled to the surface of the proximal end portion of the screw.

In some embodiments, an apparatus includes a spine stabilizer having a proximal end portion and a distal end portion. The spine stabilizer can be used, for example, to dynamically stabilize a portion of the spine to treat spinal stenosis. The distal end portion of the spine stabilizer is configured to be threaded into a bone tissue. The proximal end portion of the spine stabilizer includes a spacer having a bone engagement surface. The bone engagement surface has a first shape and is spaced apart from a longitudinal axis of the spine stabilizer by a first distance when the spine stabilizer is in a first configuration. The bone engagement surface has a second shape and is spaced apart from the longitudinal axis of the spine stabilizer by a second distance when the spine stabilizer is in a second configuration. In some embodiments, for example, the spacer can move radially relative to the longitudinal axis when the spine stabilizer is moved between the first configuration and the second configuration. The second distance greater than the first distance, and the second shape substantially the same as the first shape.

In some embodiments, an apparatus includes a bone screw and a spacer movably coupled to a proximal end portion of the bone screw. A distal end portion of the bone screw is configured to be threaded into a bone tissue. The spacer has a bone engagement surface and defines a first opening and a second opening. The first opening is configured to receive at least a portion of the proximal end portion of the bone screw. The second opening is configured to receive a portion of an insertion tool.

In some embodiments, a method includes inserting a bone stabilizer into a body. The bone stabilizer includes a bone screw, a spacer coupled to a proximal end portion of the bone screw, and a retention screw. In some embodiments, for example, the inserting includes threading the bone screw into a pedicle of an S1 vertebra. The spacer is moved relative to the bone screw from a first position to a second position using an actuation tool. The retention screw is rotated relative to the bone screw using the actuation tool such that the spacer is maintained in the second position. In some embodiments, for example, when the spacer is in the second position, a bone engagement surface of the spacer is configured to contact a portion of an inferior articulate process of an L5 vertebra.

As used in this specification, the words “proximal” and “distal” refer to the direction closer to and away from, respectively, an operator (e.g., surgeon, physician, nurse, technician, etc.) who would insert a medical device into the patient, with the tip-end (i.e., distal end) of the device inserted inside a patient's body first. Thus, for example, the end of a medical device first inserted into the patient's body is the distal end, while the opposite end of the medical device (i.e., the end of the medical device last inserted into to the patient's body and/or the end of the medical device being manipulated by the operator) is the proximal end of the medical device.

The term “parallel” is used herein to describe a relationship between two geometric constructions (e.g., two lines, two planes, a line and a plane, two curved surfaces, a line and a curved surface or the like) in which the two geometric constructions are substantially non-intersecting as they extend substantially to infinity. For example, as used herein, a planar surface (i.e., a two-dimensional surface) is said to be parallel to a line when every point along the line is spaced apart from the nearest portion of the surface by a substantially equal distance. Similarly, a line is said to be parallel to a curved surface when the line and the curved surface do not intersect as they extend to infinity and when every point along the line is spaced apart from the nearest portion of the curved surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

The terms “perpendicular,” “orthogonal,” and/or “normal” are used herein to describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal to a curved surface when the line and a portion of the curved surface intersect at an angle of approximately 90 degrees within a plane. Two geometric constructions are described herein as being, for example, “perpendicular” or “substantially perpendicular” to each other when they are nominally perpendicular to each other, such as for example, when they are perpendicular to each other within a tolerance. Such tolerances can include, for example, manufacturing tolerances, measurement tolerances or the like.

As used herein the term “sacral vertebra” refers to a vertebra associated with a sacrum of a spinal column. For example, the sacrum includes five vertebra fused together, referred to as the S1, S2, S3, S4, and S5 sacral vertebrae. The S1 sacral vertebra is superior to the S2 sacral vertebra, the S2 sacral vertebra is superior to the S3 sacral vertebra and so on. As used herein the term “lumbar vertebra” refers to the L1-L5 vertebrae of the spinal column, with the L5 lumbar vertebra being superior to the S1 sacral vertebra, the L4 lumbar vertebra being superior to the L5 vertebra, the L3 vertebra being superior to the L4 vertebra and so on. As used herein, the terms “vertebra” and “vertebrae” used without a modifier can refer to any type of vertebra or vertebrae (e.g., sacral, lumbar, thoracic, cervical).

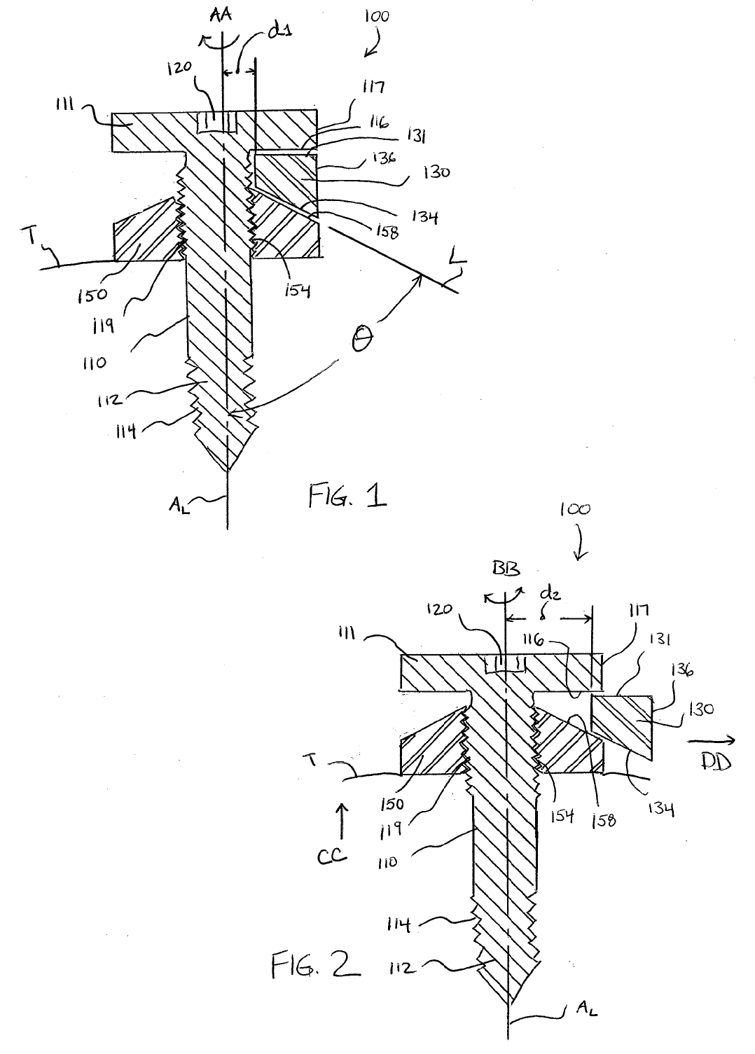

FIGS. 1 and 2 are schematic illustrations of a cross-section of a bone fixation device 100 according to an embodiment, in a first configuration (FIG. 1) and a second configuration (FIG. 2). The bone fixation device 100 includes a screw 110, an actuator 150 and a spacer 130. The screw 110 includes a proximal end portion 111 and a distal end portion 112, and defines a longitudinal axis A_{L}. The proximal end portion 111 of the screw 110 includes a threaded portion 119 and a tool engagement opening 120. The tool engagement opening 120 is configured to receive and/or engage a portion of an insertion tool (not shown in FIGS. 1 and 2). The tool engagement opening 120 can be, for example, a hexagonal-shaped recess corresponding to a hexagonal-shaped tip of the insertion tool. In this manner, the tool engagement opening 120 of the screw 110 can receive a portion of the insertion tool such that rotation of the insertion tool results in rotation of the screw 110 about the longitudinal axis A_{L}, as shown by the arrow AA in FIG. 1. The distal end portion 112 includes a threaded portion 114 such that the distal end portion 112 of the screw 110 can be threaded into a bone tissue. The threaded portion 114 can include, for example, a self-tapping tip.

As described in more detail herein, the actuator 150 is configured to move the spacer 130 relative to the screw 110 between a first position (FIG. 1) and a second position (FIG. 2). The actuator 150 includes a first surface 158 and a threaded portion 154. The threaded portion 154 of the actuator 150 corresponds to (i.e., has substantially the same nominal size and thread pitch) the threaded portion 119 of the proximal end portion 111 of the screw 110. Similarly stated, the threaded portion 154 of the actuator 150 includes female threads that correspond to the male threads of the threaded portion 119 of the screw 110. In this manner, the actuator 150 is threadedly coupled to the screw 110. More particularly, the proximal end portion 111 of the screw 110 is disposed within the actuator 150 such that the threaded portion 119 of the screw 110 is engaged with the threaded portion 154 of the actuator 150. In this manner, rotation of the actuator 150 relative to the screw 110 results in movement of the actuator 150 relative to the screw 110 along the longitudinal axis A_{L}.

The first surface 158 of the actuator 150 defines a line L that is offset from the longitudinal axis A_{L} by an angle Θ having a value less than approximately 90 degrees and greater than approximately 0 degrees. Said another way, the first surface 158 of the actuator 150 defines a line L that is substantially non-parallel to and substantially non-normal to the longitudinal axis A_{L}. In some embodiments, for example, the first surface 158 can have a frusto-conical shape, and the line L can extend radially from a center portion of the actuator 150 towards the outer edge of the actuator 150. In other embodiments, the first surface 158 can define a plane, within which the line L is defined.

The spacer 130 includes a first surface 131, a second surface 134 and a third surface 136. As shown in FIGS. 1 and 2, the spacer 130 is disposed between the screw 110 and the actuator 150. The first surface 131 of the spacer 130 is substantially parallel to and in contact with a surface 116 of the proximal end portion 111 of the screw 110 (the first surface 131 is shown as being spaced apart from the surface 116 in FIGS. 1 and 2 for purposes of clarity). The second surface 134 of the spacer 130 is substantially parallel to and in contact with the first surface 158 of the actuator 150 (the second surface 134 is shown as being spaced apart from the first surface 158 in FIGS. 1 and 2 for purposes of clarity). Similarly stated, a line (not shown in FIGS. 1 and 2) defined by the second surface 134 of the spacer 130 is substantially parallel to the line L defined by the first surface 158 of the actuator.

As shown in FIGS. 1 and 2, the bone fixation device 100 is movable between a first configuration (FIG. 1) and a second configuration (FIG. 2). When the bone fixation device 100 is in the first configuration, the spacer 130 is spaced apart from the longitudinal axis A_{L} by a first distance d1. Although the spacer 130 is shown as being spaced apart from the shank of the screw 110 when the bone fixation device 100 is in the first configuration, in other embodiments, the spacer 130 can be in contact with the shank of the screw 110 when the bone fixation device 100 is in the first configuration. Although the third surface 136 of the spacer 130 is shown as being substantially aligned with an outer surface 117 of the proximal end portion 111 of the screw 110 when the bone fixation device 100 is in the first configuration, in other embodiments, the third surface 136 of the spacer 130 can be out of alignment with the outer surface 117 of the proximal end portion 111 of the screw 110 when the bone fixation device 100 is in the first configuration. Similarly stated, although the third surface 136 of the spacer 130 is shown as being substantially flush with the outer surface 117 of the proximal end portion 111 of the screw 110 when the bone fixation device 100 is in the first configuration, in other embodiments, the third surface 136 of the spacer 130 and the outer surface 117 of the proximal end portion 111 of the screw 110 can form a discontinuous surface when the bone fixation device 100 is in the first configuration.

To move the bone fixation device 100 to the second configuration, the screw 110 is rotated relative to the actuator 150 (and/or the actuator 150 is rotated relative to the screw 110) as shown by the arrow BB in FIG. 2. Rotation of the actuator 150 relative to the screw 110 results in movement of the actuator 150 relative to the screw along the longitudinal axis A_{L}, as shown by the arrow CC in FIG. 2. The axial movement of the actuator 150 causes the first surface 158 of the actuator 150 to exert an axial force (i.e., a force in the direction shown by the arrow CC) on the second surface 134 of the spacer 130. Because the first surface 158 of the actuator 150 is offset from the longitudinal axis A_{L} by the angle θ, a component of the axial force transmitted via the first surface 158 of the actuator 150 to the second surface 134 of the spacer 130 has a radial direction as shown by the arrow DD in FIG. 2. Said another way, a component of the force exerted by the actuator 150 on the spacer 130 has a direction that is substantially normal to the longitudinal axis A_{L}. Accordingly, the force exerted by the actuator 150 on the spacer 130 causes the second surface 134 of the spacer 130 to slide on the first surface 158 of the actuator 150, and causes the spacer 130 to move in the direction shown by the arrow DD in FIG. 2.

As shown in FIG. 2, when the bone fixation device 100 is in the second configuration, the spacer 130 is spaced apart from the longitudinal axis A_{L} by a second distance d2, which is greater than the first distance d1. When the bone fixation device 100 is in the second configuration, the spacer 130 is spaced apart from the shank of the screw 110, and the third surface 136 of the spacer 130 is out of alignment with the outer surface 117 of the proximal end portion 111 of the screw 110. Similarly stated, when the bone fixation device 100 is in the second configuration, the third surface 136 of the spacer 130 and the outer surface 117 of the proximal end portion 111 of the screw 110 forms a discontinuous surface. In this manner, as described in more detail herein, the third surface 136 of the spacer 130 can be disposed against a bone tissue (not shown in FIGS. 1 and 2) to stabilize the bone tissue.

The angle θ of the first surface 158 of the actuator 150 can be any suitable angle between 0 and 90 degrees. The value of the angle θ can affect the force used to move the bone fixation device 100 from the first configuration to the second configuration and/or the distance through which the spacer 130 travels when the bone fixation device 100 is moved from the first configuration to the second configuration. More particularly, if the angle θ is close to 0 degrees, the force to move the implant 100 from the first configuration to the second configuration will be less than the force needed if the angle θ is close to 90 degrees. Said another way, when the first surface 158 of the actuator 150 is close to being parallel to the longitudinal axis A_{L}, less force is needed to move the bone fixation device 100 to the second configuration than when the first surface 158 of the actuator 150 is close to being normal to the longitudinal axis A_{L}.

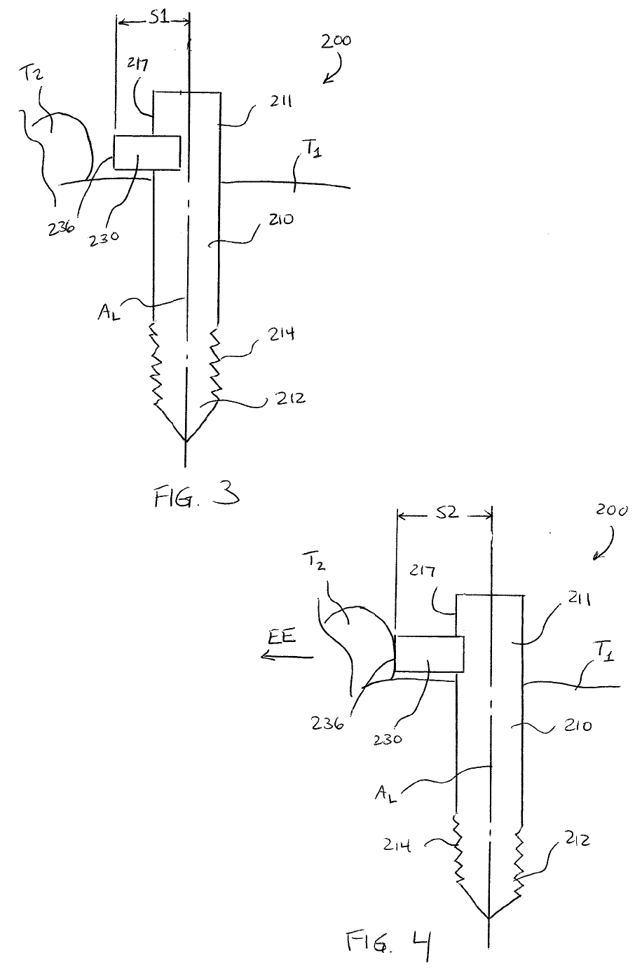

FIGS. 3 and 4 are schematic illustrations of a spine stabilizer 200, according to an embodiment, in a first configuration (FIG. 3) and a second configuration (FIG. 4). The spine stabilizer 200 includes a screw 210 and a spacer 230. The screw 210 includes a proximal end portion 211 and a distal end portion 212, and defines a longitudinal axis A_{L}. The distal end portion 212 of the screw 210 includes a threaded portion 214 such that the distal end portion 212 of the screw 210 can be threaded into a first portion of a bone structure T₁. The threaded portion 214 can include, for example, a self-tapping tip. The first portion of the bone structure T1 can be, for example, a pedicle of a vertebra. In some embodiments, the screw 210 can include a tool engagement portion (not shown in FIGS. 3 and 4) configured to receive and/or engage a portion of an insertion tool (not shown in FIGS. 3 and 4), as described above.

The spacer 230 is coupled to the proximal end portion 211 of the screw 210, and has a bone engagement surface 236. As shown in FIGS. 3 and 4, the spine stabilizer 200 is movable between a first configuration (FIG. 3) and a second configuration (FIG. 4). When the spine stabilizer 200 is in the first configuration, the bone engagement surface 236 of the spacer 230 is spaced apart from the longitudinal axis A_{L} by a first distance S1. Although the first distance S1 is shown as being non-zero, in other embodiments, the first distance S1 can be zero. Similarly stated, the spine stabilizer 200 has a maximum size S1 taken in a direction substantially normal to the longitudinal axis A_{L} when the spine stabilizer 200 is in the first configuration. Although the bone engagement surface 236 of the spacer 230 is shown as being misaligned with an outer surface 217 of the proximal end portion 211 of the screw 210 when the spine stabilizer 200 is in the first configuration, in other embodiments, the bone engagement surface 236 of the spacer 230 is substantially aligned with the outer surface 217 of the proximal end portion 211 of the screw 210 when the spine stabilizer 200 is in the first configuration. Similarly stated, in some embodiments, the bone engagement surface 236 of the spacer 230 can be substantially flush with the outer surface 217 of the proximal end portion 211 of the screw 210 when the spine stabilizer 200 is in the first configuration.

When the spine stabilizer 200 is in the first configuration, the bone engagement surface 236 and/or the spacer 230 has a first shape. In some embodiments, for example, the bone engagement surface 236 can have a substantially rectangular shape when the spine stabilizer 200 is in the first configuration. In other embodiments, the bone engagement surface 236 can have a first shape that corresponds to a shape of a second portion of the bone structure T₂. For example, in some embodiments, the first shape can be concave such that the bone engagement surface 236 forms a saddle to receive the second portion of the bone structure T₂.

To move the spine stabilizer 200 to the second configuration, the spacer 230 is moved relative to the screw 210, as shown by the arrow EE in FIG. 4. The spacer 230 can be moved relative to the screw 210 by any suitable mechanism. For example, in some embodiments, the spacer 230 can be moved relative to the screw 210 by a mechanical actuator (not shown in FIGS. 3 and 4) such as the types shown and described herein. In other embodiments, the spacer 230 can be moved relative to the screw 210 by an electronic actuator, a magnetic actuator, a hydraulic actuator and/or a pneumatic actuator. For example, in some embodiments, the proximal end portion 211 of the screw 210 can include a magnetic portion configured to selectively attract and/or repel the spacer 230 to move the spine stabilizer 200 between the first configuration and the second configuration. In other embodiments, a portion of the screw 210 can be pressurized with a fluid (e.g., a liquid or a gas) to cause the spacer 230 to move relative to the screw 210.

As shown in FIG. 4, when the spine stabilizer 200 is in the second configuration, when the spine stabilizer 200 is in the second configuration, the bone engagement surface 236 of the spacer 230 is spaced apart from the longitudinal axis A_{L} by a second distance S2. Similarly stated, the spine stabilizer 200 has a maximum size S2 taken in a direction substantially normal to the longitudinal axis A_{L} when the spine stabilizer 200 is in the second configuration. As shown in FIG. 4, the size S2 is greater than the size S1.

When the spine stabilizer 200 is in the second configuration, the bone engagement surface 236 and/or the spacer 230 has a second shape that is substantially the same as the first shape. Said another way, when the spine stabilizer 200 is moved from the first configuration to the second configuration, the shape of the bone engagement surface 236 and/or the spacer 230 remains substantially unchanged. Similarly stated, the bone engagement surface 236 and/or the spacer 230 are not substantially deformed when the spine stabilizer 200 is moved from the first configuration to the second configuration. In other embodiments, however, the spacer 230 can be deformed when the spine stabilizer 200 is moved from the first configuration to the second configuration.

As described in more detail below, the spine stabilizer 200 can be used to secure and/or stabilize tissue within the body. More particularly, in some embodiments, the spine stabilizer 200 can be used to stabilize a portion of a spinal column. For example, as shown in FIG. 3, the spine stabilizer 200 can be coupled to a first portion of a bone structure T₁ (e.g., a pedicle of a first vertebra) via the threaded portion 214 of the screw 210 when the spine stabilizer is in the first configuration. The spine stabilizer 200 can then be moved from the first configuration to the second configuration. As shown in FIG. 4, when the spine stabilizer 200 is in the second configuration, the bone engagement surface 236 of the spacer 230 can contact, engage and/or exert a force upon the second portion of the bone structure T2 (e.g., an inferior articulate process of a second vertebra). In this manner, the second portion of the bone structure T₂ can be moved, stabilized and/or secured relative to the first portion of the bone structure T₁. Similarly stated, movement of the second portion of the bone structure T₂ relative to the first portion of the bone structure T₁ can be limited.

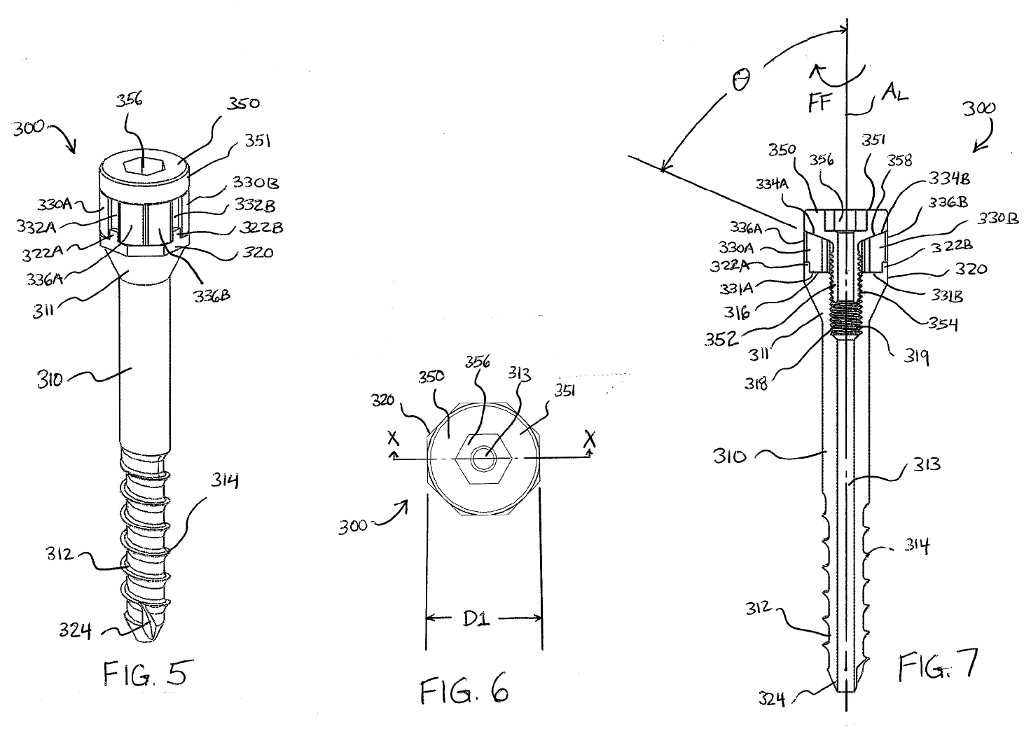

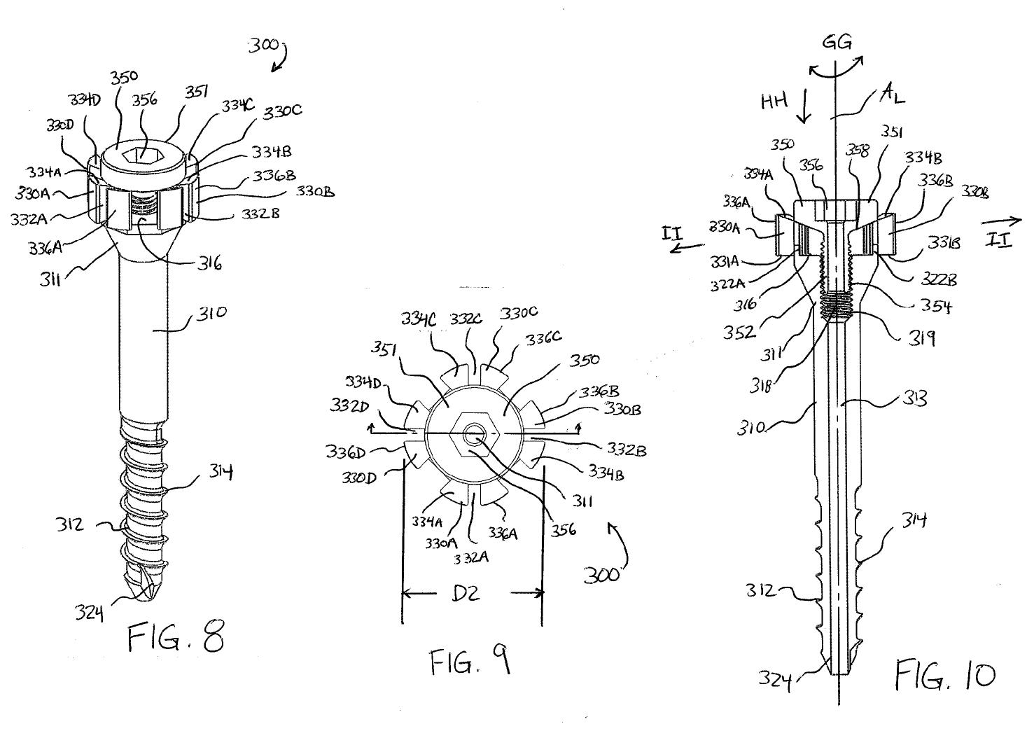

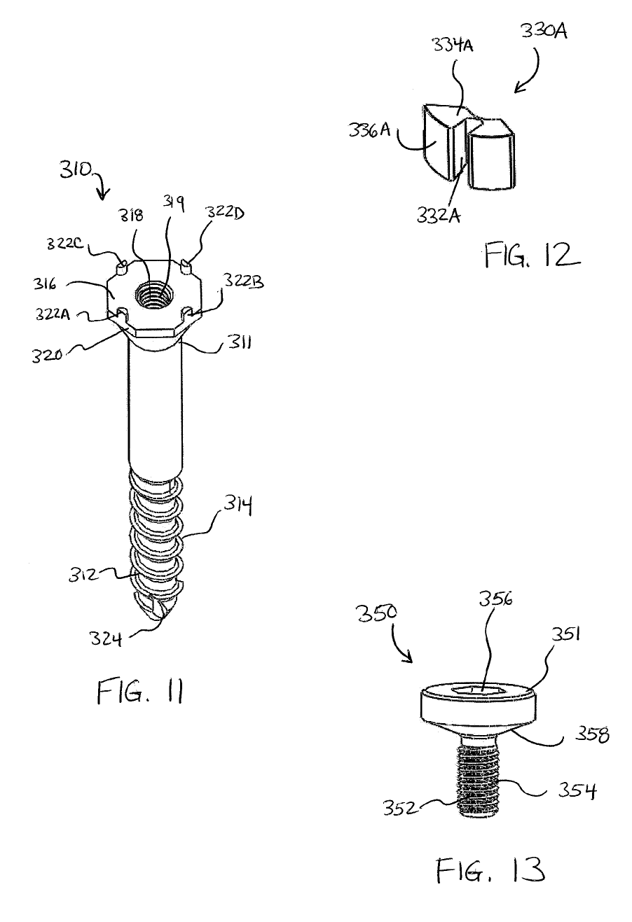

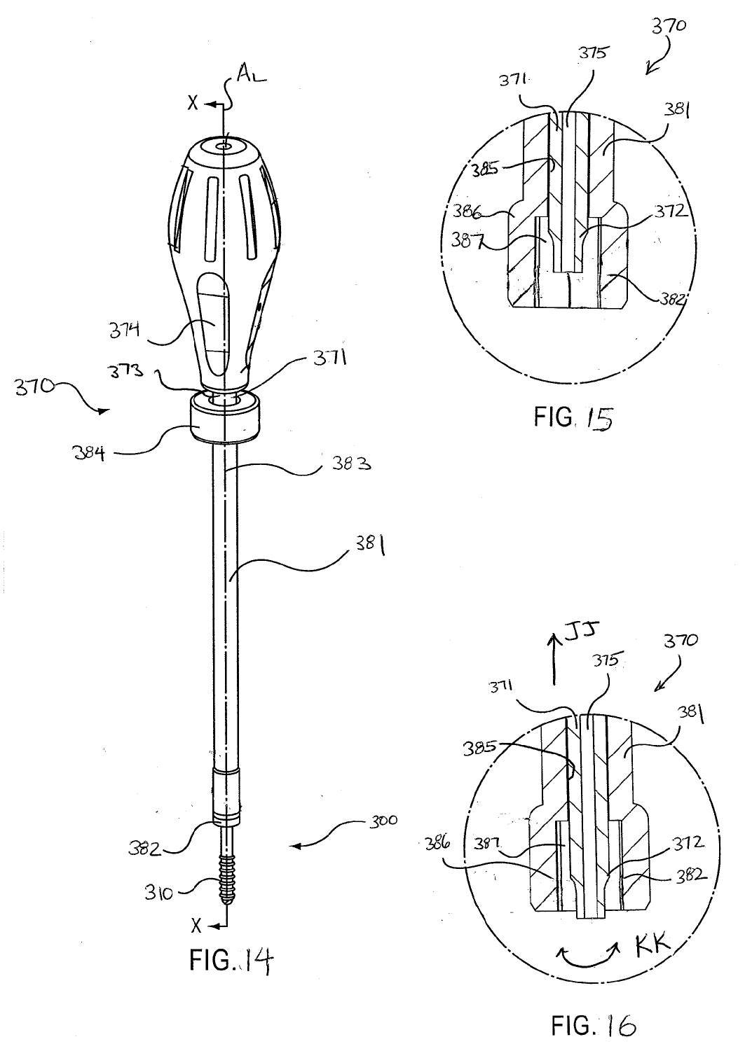

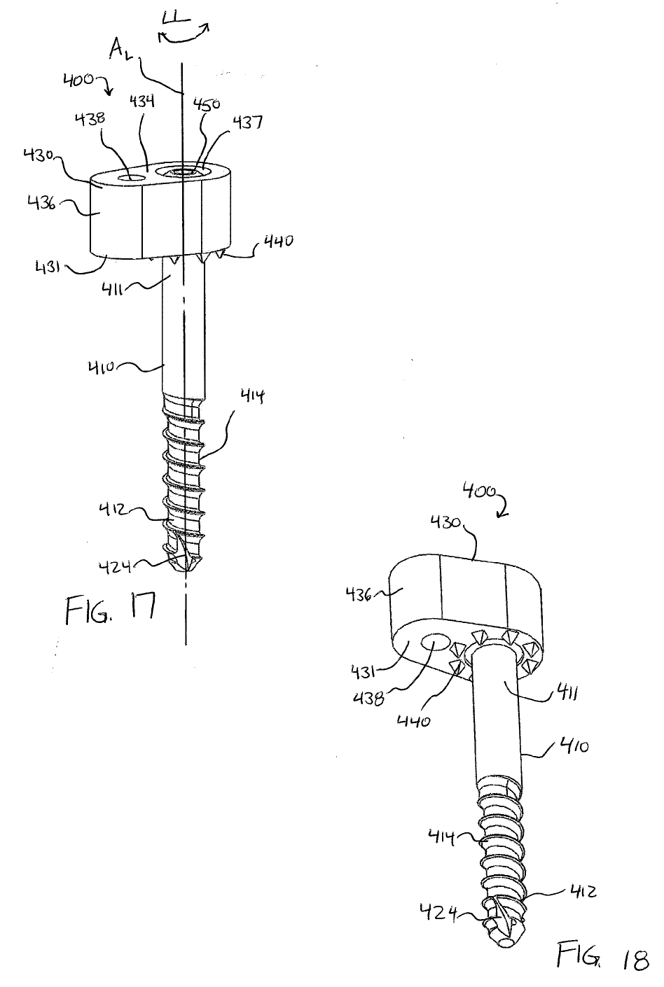

FIGS. 5-13 show a bone fixation device 300 according to an embodiment. FIGS. 5-7 show the bone fixation device 300 in a first configuration and FIGS. 8-10 show the bone fixation device 300 in a first configuration. FIGS. 11-13 shows portions of the bone fixation device 300. The bone fixation device 300 includes a screw 310, an actuator 350 and four spacers 330A, 330B, 330C and 330D. The screw 310 includes a proximal end portion 311 and a distal end portion 312, and defines a longitudinal axis A_{L}. The distal end portion 312 of the screw 310 includes a threaded portion 314 and a self-tapping tip 324. In this manner, the screw 310 can be threaded into a bone tissue, as described in more detail herein. As shown in FIGS. 7 and 10, the screw 310 defines a lumen 313 therethrough. Similarly stated, the screw 310 is a cannulated screw that can be disposed about a guide member, such as, for example, a guide wire, a Kirschner wire (i.e., a K-wire) and/or the like.

As shown in FIGS. 7, 8, 10 and 11, the proximal end portion 311 of the screw 310 includes an end surface 316 and a side surface 320. As shown in FIG. 11, the end surface 316 includes four protrusions 322A, 322B, 322C and 322D. As described in more detail herein, the protrusions 322A, 322B, 322C and 322D are disposed within the corresponding grooves 332A, 332B, 332C and 332D (see e.g., FIG. 9) to limit the movement of the spacers 330A, 330B, 330C and 330D relative to the screw 310. As shown in FIGS. 7, 10 and 11, the end surface 316 defines an opening 318 that includes a threaded portion 319. Although the opening 318 is shown as being substantially coaxial with the longitudinal axis A_{L} and/or the lumen 313, in other embodiments the opening 318 can be offset from the longitudinal axis A_{L} and/or the lumen 313.

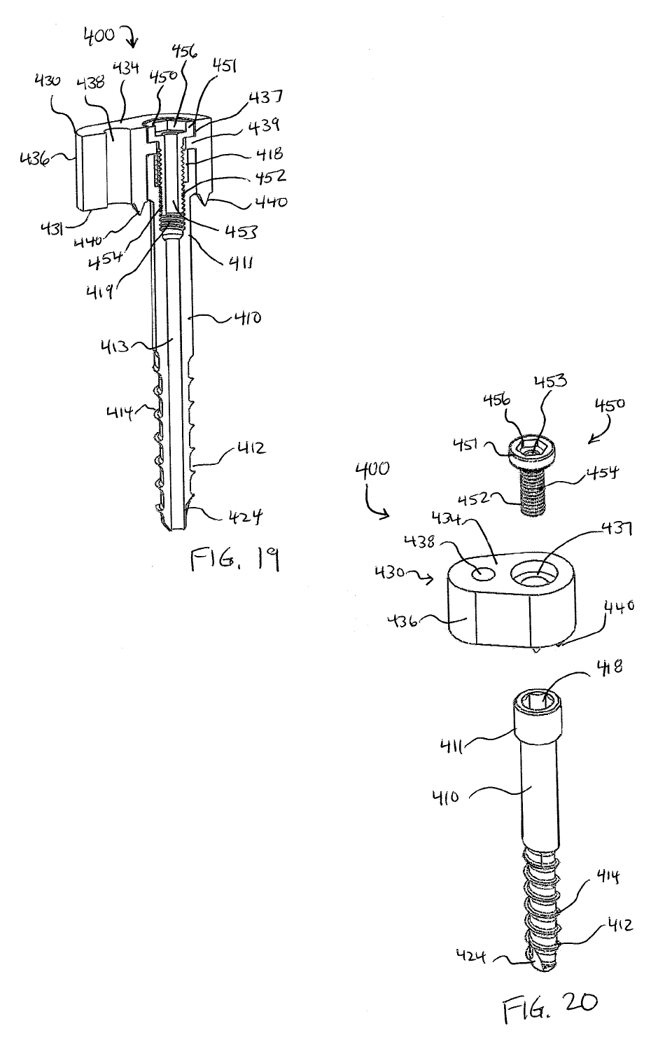

The side surface 320 is configured to engage a portion of an insertion and/or adjustment tool, such as, for example, the insertion tool 370 shown in FIGS. 14-16. More specifically, the side surface 320 is an eight-sided surface that is configured to be received within a corresponding opening 387 of the insertion tool 370. In this manner, when the proximal end portion 311 of the screw 310 is engaged with the insertion tool 370, rotation of an outer shaft 381 of the insertion tool 370 results in rotation of the screw 310 about the longitudinal axis A_{L}, as shown by the arrow FF in FIG. 7. Thus, as described in more detail below, the insertion tool 370 can be used to advance (e.g., thread) the screw 310 into a bone tissue. In some embodiments, the side surface 320 can be configured to limit the axial movement of the screw 310 with respect to the insertion tool. For example, in some embodiments, the side surface 320 can define openings and/or grooves configured to receive a snap ring, clip, E-ring or any other suitable mechanism for removably coupling the screw 310 to the insertion and/or adjustment tool. Although the side surface 320 is shown as being an eight-sided surface, in other embodiments, the side surface 320 can have any suitable shape (e.g., a hexagonal shape, a rectangular shape or the like).

As described in more detail herein, the actuator 350 is configured to move the spacers 330A, 330B, 330C and 330D relative to the screw 310 between a first position (FIGS. 5-7) and a second position (FIGS. 8-10). The actuator 350 has a proximal end portion 351 and a distal end portion 352, and defines a lumen 353 therethrough. The distal end portion 352 includes a threaded portion 354 that corresponds to (i.e., has substantially the same nominal size and thread pitch) the threaded portion 319 of the proximal end portion 311 of the screw 310. Similarly stated, the threaded portion 354 of the actuator 350 includes male threads that correspond to the female threads within the opening 318 of the screw 310. Thus, the actuator 350 is threadedly coupled to the screw 310 such that that the lumen 353 of the actuator 350 is substantially coaxial with the longitudinal axis A_{L}. In this arrangement, rotation of the actuator 350 relative to the screw 310 results in movement of the actuator 350 relative to the screw 310 along the longitudinal axis A_{L}.

The proximal end portion 351 of the actuator 350 includes an actuation surface 358 and defines a tool engagement opening 356. The tool engagement opening 356 is configured to receive and/or engage a portion of an insertion and/or adjustment tool, such as, for example, the insertion tool 370 shown in FIGS. 14-16. More specifically, the tool engagement opening 356 is a hexagonally-shaped recess configured to receive a corresponding tip 372 of the insertion tool 370. In this manner, when the proximal end portion 351 of the actuator 350 is engaged with the insertion tool 370, rotation of the inner shaft 371 of the insertion tool 370 results in rotation of the actuator 350 about the longitudinal axis A_{L}, as shown by the arrow GG in FIG. 10. Thus, as described in more detail below, the insertion tool 370 can be used to move the actuator 350 relative to the screw 310.

As shown in FIG. 7, the actuation surface 358 of the actuator 350 defines a line L that is offset from the longitudinal axis A_{L} by an angle Θ having a value less than approximately 90 degrees and greater than approximately 0 degrees. Said another way, the actuation surface 358 defines a line L that is substantially non-parallel to and substantially non-normal to the longitudinal axis A_{L}. More particularly, the first surface 358 has a frusto-conical shape, and the line L extends radially from a center portion of the actuator 350 towards the outer edge of the actuator 350. Thus, the actuation surface 358 is a tapered and/or ramped surface.

Each of the spacers 330A, 330B, 330C and 330D includes a first surface 331A, 331B, 331C and 331D (see e.g., FIGS. 7 and 10), a second surface 334A, 334B, 334C and 334D, and a third surface 336A, 336B, 336C and 336D. The third surface 336A, 336B, 336C and 336D of each spacer 330A, 330B, 330C and 330D defines a groove 332A, 332B, 332C and 332D. The spacers 330A, 330B, 330C and 330D are disposed between the screw 310 and the actuator 350 such that the first surface 331A, 331B, 331C and 331D of each spacer 330A, 330B, 330C and 330D is substantially parallel to and in contact with the end surface 316 of the proximal end portion 311 of the screw 310. The second surface 334A, 334B, 334C and 334D of each spacer 330A, 330B, 330C and 330D is substantially parallel to and in contact with the first surface 358 of the actuator 350. Similarly stated, a line (not shown in FIGS. 7 and 10) defined by the second surface 334A, 334B, 334C and 334D of each spacer 330A, 330B, 330C and 330D is substantially parallel to the line L defined by the first surface 358 of the actuator.

Moreover, the spacers 330A, 330B, 330C and 330D are disposed between the screw 310 and the actuator 350 such that the protrusions 322A, 322B, 322C and 322D of the screw 310 are disposed within the corresponding grooves 332A, 332B, 332C and 332D of each spacer 330A, 330B, 330C and 330D. Thus, the first surface 331A, 331B, 331C and 331D of each spacer 330A, 330B, 330C and 330D is matingly and movably coupled to the end surface 316 of the proximal end portion 311 of the screw 310. In this manner, as described in more detail below, the protrusions 322A, 322B, 322C and 322D and the grooves 332A, 332B, 332C and 332D can cooperatively allow the spacers 330A, 330B, 330C and 330D to move radially a predetermined distance relative to the actuator 350 and/or the screw 310. Said another way, the protrusions 322A, 322B, 322C and 322D and the side wall defining the grooves 332A, 332B, 332C and 332D are cooperatively configured to limit the radial movement of the spacers 330A, 330B, 330C and 330D relative to the actuator 350 and/or the screw 310.

The bone fixation device 300 is movable between the first configuration (FIGS. 5-7) and a second configuration (FIGS. 8-10). When the bone fixation device 300 is in the first configuration, the third surface 336A of the spacer 330A and the third surface 336B of the spacer 330B are each spaced apart from the longitudinal axis A_{L} by a first distance. Said another way, as shown in FIG. 6, the bone fixation device 300 has a maximum outer diameter D1 when the bone fixation device 300 is in the first configuration. Although the third surfaces 336A, 336B, 336C and 336D of the spacers 330A, 330B, 330C and 330D are shown as being substantially aligned with at least a portion of the side surface 320 of the screw 310 and/or a side surface of the actuator 350 when the bone fixation device 300 is in the first configuration, in other embodiments, the third surfaces 336A, 336B, 336C and 336D of the spacers 330A, 330B, 330C and 330D can be out of alignment with the side surface 320 of the screw 310 and/or the side surface of the actuator 350 when the bone fixation device 300 is in the first configuration. Similarly stated, although the third surfaces 336A, 336B, 336C and 336D of the spacers 330A, 330B, 330C and 330D are shown as being substantially flush with at least a portion of the side surface 320 of the screw 310 and/or the side surface of the actuator 350 when the bone fixation device 300 is in the first configuration, in other embodiments, the third surfaces 336A, 336B, 336C and 336D of the spacers 330A, 330B, 330C and 330D can form a discontinuous surface with the side surface 320 of the screw 310 and/or the side surface of the actuator 350 when the bone fixation device 300 is in the first configuration.

To move the bone fixation device 300 to the second configuration, the actuator 350 is rotated relative to the screw 310 as shown by the arrow GG in FIG. 10. Rotation of the actuator 350 relative to the screw 310 results in movement of the actuator 350 relative to the screw along the longitudinal axis A_{L}, as shown by the arrow HH in FIG. 10. The axial movement of the actuator 350 causes the first surface 358 of the actuator 350 to exert an axial force (i.e., a force in the direction shown by the arrow HH) on the second surface 334A, 334B, 334C and 334D of the spacers 330A, 330B, 330C and 330D. Because the first surface 358 of the actuator 350 is offset from the longitudinal axis A_{L} by the angle θ, a component of the axial force transmitted via the first surface 358 of the actuator 350 to the second surface 334A, 334B, 334C and 334D of the spacers 330A, 330B, 330C and 330D has a radial direction as shown by the arrows II in FIG. 10. Said another way, a component of the force exerted by the actuator 350 on the spacers 330A, 330B, 330C and 330D has a direction that is substantially normal to the longitudinal axis A_{L}. Accordingly, the force exerted by the actuator 350 on the spacer 330 causes the second surface 334A, 334B, 334C and 334D of the spacers 330A, 330B, 330C and 330D to slide on the first surface 358 of the actuator 350, and causes the spacer 330 to move in the direction shown by the arrows II in FIG. 10.

As shown in FIG. 10, the actuator 350 moves longitudinally relative to the screw 310 when the bone fixation device 300 is moved between the first configuration and the second configuration. Similarly stated, the actuator 350 moves relative to the screw 310 through a range of motion when the bone fixation device 300 is moved between the first configuration and the second configuration. Moreover, the spacers 330A, 330B, 330C and 330D each move radially through a range of motion relative to the screw 310 when the bone fixation device 300 is moved between the first configuration and the second configuration. As shown in FIGS. 7 and 10, the second surface 334A, 334B, 334C and 334D of each spacer 330A, 330B, 330C and 330D is substantially parallel to and in contact with the first surface 358 of the actuator 350 throughout the range of motion of the actuator 350 and/or the range of motion of the spacers 330A, 330B, 330C and 330D. Said another way, when viewed as a two-dimensional cross-section, the line (not shown in FIGS. 7 and 10) defined by the second surface 334A, 334B, 334C and 334D of each spacer 330A, 330B, 330C and 330D is substantially parallel to the line L defined by the first surface 358 of the actuator throughout the range of motion of the actuator 350 and/or the range of motion of the spacers 330A, 330B, 330C and 330D. Similarly stated, the orientation of the spacers 330A, 330B, 330C and 330D relative to the actuator 350 remains substantially constant when the bone fixation device 300 is moved between the first configuration and the second configuration.

When the bone fixation device 300 is in the second configuration, the third surface 336A of the spacer 330A and the third surface 336B of the spacer 330B are each spaced apart from the longitudinal axis A_{L} by a second distance. Said another way, as shown in FIG. 9, the bone fixation device 300 has a maximum outer diameter D2 greater than the outer diameter D1 when the bone fixation device 300 is in the second configuration. The outer diameter D2 can be greater than the outer diameter D1 by any suitable amount. For example, in some embodiments, the outer diameter D2 can be greater than the outer diameter D1 by between 1 and 2 millimeters.

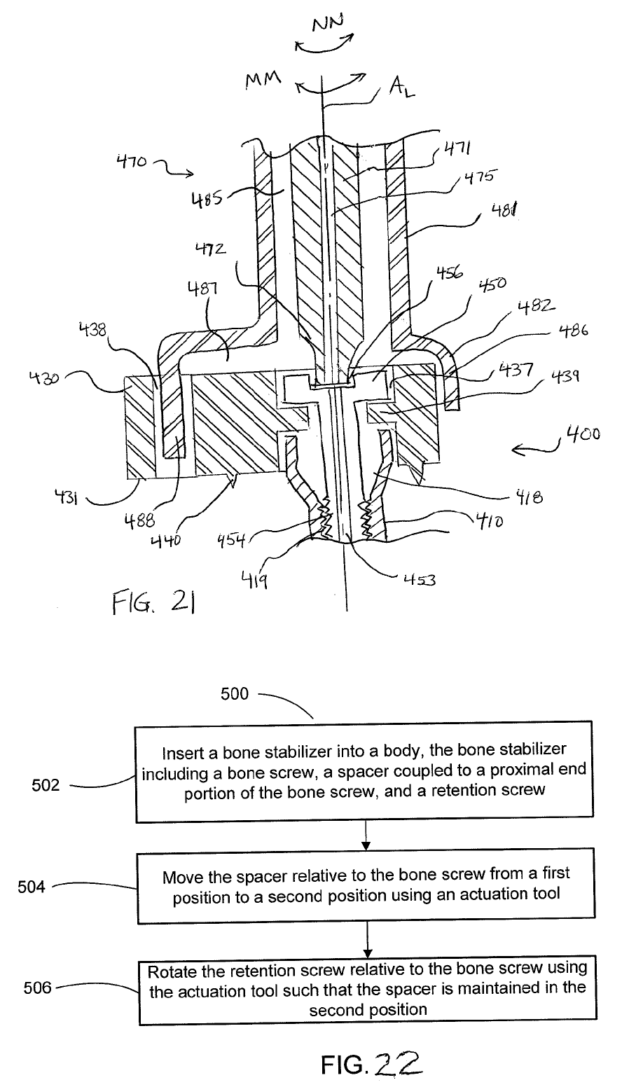

When the bone fixation device 300 is in the second configuration, the third surfaces 336A, 336B, 336C and 336D of the spacers 330A, 330B, 330C and 330D are out of alignment with at least a portion of the side surface 320 of the screw 310 and/or the side surface of the actuator 350. Similarly stated, when the bone fixation device 300 is in the second configuration, the third surfaces 336A, 336B, 336C and 336D of the spacers 330A, 330B, 330C and 330D and the side surface 320 of the screw 310 and/or the side surface of the actuator 350 form a discontinuous surface. In this manner, as described in more detail herein, at least one of the third surfaces 336A, 336B, 336C and 336D of the spacers 330A, 330B, 330C and 330D can be disposed against a bone tissue (see e.g., FIG. 18) to stabilize and/or limit the movement of the bone tissue relative to the implant 300.

The angle θ of the first surface 358 of the actuator 350 can be any suitable angle between 0 and 90 degrees. The value of the angle θ can affect the force used to move the bone fixation device 300 from the first configuration to the second configuration and/or the distance through which the spacer 330 travels when the bone fixation device 300 is moved from the first configuration to the second configuration. More particularly, if the angle θ is close to 0 degrees, the force to move the implant 300 from the first configuration to the second configuration will be less than the force needed if the angle θ is close to 90 degrees. Said another way, when the first surface 358 of the actuator 350 is close to being parallel to the longitudinal axis A_{L}, less force is needed to move the bone fixation device 300 to the second configuration than when the first surface 358 of the actuator 350 is close to being normal to the longitudinal axis A_{L}.

The bone fixation device 300 can be inserted into a body (not shown) using the insertion tool 370 shown in FIGS. 14-16. The insertion tool 370 includes an outer shaft 381 and an inner shaft 371 disposed within the outer shaft 381. The outer shaft 381, which can also be referred to as the nut driver shaft, includes a proximal end portion 383 and a distal end portion 382. The outer shaft 381 defines a lumen 385 therethrough (see FIGS. 15 and 16) and a longitudinal axis A_{L}.

The proximal end portion 383 of the outer shaft 381 includes an actuator 384 configured to be manipulated by a user to move the outer shaft 381. More particularly, the actuator 384 is configured to be grasped and/or manipulated by the user to rotate the outer shaft 381 about the inner shaft 371 and/or to move the outer shaft 381 along the longitudinal axis A_{L} relative to the inner shaft 371. The outer surface of the actuator 384 can include any suitable topographical features to aid in the manipulation of the outer shaft 381. For example, in some embodiments, the outer surface of the actuator 384 can be knurled, cross-hatched or the like.

The distal end portion 382 of the outer shaft 381 includes a side wall 386 that defines an opening 387. The opening 387 is configured to receive the proximal end portion of the bone fixation device 300 when the insertion tool 370 is in a first configuration (shown in FIG. 15) and when the bone fixation device 300 is in the first configuration. More specifically, the inner surface of the side wall 386 includes a series of flats corresponding to the side surface 320 of the screw 310. In this manner, when the proximal end portion 311 of the screw 310 is disposed within the opening 387, rotation of the outer shaft 381 of the insertion tool 370 results in rotation of the screw 310 about the longitudinal axis A_{L}.

The inner shaft 371, which can also be referred to as the hex driver shaft, includes a proximal end portion 373 and a distal end portion 372. The inner shaft defines a lumen 375 (see e.g., FIGS. 15 and 16) that is coaxial with the longitudinal axis A_{L}. The lumen 375 is configured to be aligned with the lumen 313 of the screw 310 when the bone fixation device 300 is engaged with the insertion tool 370. In this manner, a guide wire, a Kirschner wire or the like can be disposed through the lumen 375 of the inner shaft 371 and the lumen 313 of the screw 310 to facilitate the insertion of the bone fixation device 300 into a bone tissue.

The proximal end portion 373 of the inner shaft 371 includes a handle 374 configured to be manipulated by a user to move the inner shaft 371 and/or the outer shaft 381. More particularly, the handle 374 is configured to be grasped and/or manipulated by the user to rotate the inner shaft 371 within the outer shaft 381 and/or to move the inner shaft 371 along the longitudinal axis A_{L} relative to the outer shaft 381. The outer surface of the handle 374 can include any suitable topographical features to aid in the manipulation of the inner shaft 371.

The distal end portion 372 of the inner shaft 371 includes a set of hexagonal-shaped surfaces corresponding to the hexagonal-shaped tool engagement opening 356 defined by the actuator 350. In this manner, the distal end portion 372 of the inner shaft 371 can be received within the tool engagement opening 356 of the actuator 350 such that rotation of the inner shaft 371 about the longitudinal axis A_{L} results in rotation of the actuator 350.

The inner shaft 371 is movably disposed within the lumen 385 of the outer shaft 381. In this manner, the insertion tool 370 can be moved between a first configuration (FIG. 15) and a second configuration (FIG. 16). When the insertion tool 370 is in the first configuration, the distal end portion 372 of the inner shaft 371 is recessed within the opening 387 of the outer shaft 381. Thus, when the insertion tool 370 is in the first configuration, the proximal end of the bone fixation device 300 can be disposed within the opening 387 such that the distal end portion 372 of the inner shaft 371 is disposed within the tool engagement opening 356 of the actuator 350 and the inner surface of the side wall 386 is in contact with the side surface 320 of the screw 310. When the bone fixation device 300 is disposed within the opening 387 with the insertion tool 370 in the first configuration, the inner surface of the side wall 386 is adjacent and/or in contact with the third surfaces 336A, 336B, 336C and 336D of the spacers 330A, 330B, 330C and 330D. Thus, when the side surface 320 of the screw 310 is disposed within the opening 387, outward radial movement of the spacers 330A, 330B, 330C and 330D is limited. Said another way, when the side surface 320 of the screw 310 is disposed within the opening 387, the bone fixation device 300 cannot be moved from its first configuration to its second configuration.

When the bone fixation device 300 is disposed within the opening 387 with the insertion tool 370 in the first configuration, the insertion tool 370 can be used to advance (e.g., thread) the screw 310 into a bone tissue. More particularly, the outer shaft 381 can be rotated thereby resulting in rotation of the bone screw 310 to advance the bone screw 310 into the bone tissue. In some embodiments, the inner shaft 371 can be retained within the outer shaft 381 such that the inner shaft 371 rotates with the outer shaft 381 when the insertion tool 370 is used to advance the bone screw 310 into the bone tissue. In some embodiments, the insertion tool 370 can include a locking mechanism configured to selectively limit the rotational and/or translation movement of the inner shaft 371 within the outer shaft 381. Such a locking mechanism can be any locking mechanism such as the types shown and described in U.S. patent application Ser. No. 12/112,650 entitled “Apparatus and Methods for Inserting Facet Screws,” filed Apr. 30, 2008, which is incorporated herein by reference in its entirety.

After the bone screw 310 is advanced into the bone tissue, the outer shaft 381 can be moved longitudinally relative to the inner shaft 371, as shown by the arrow JJ in FIG. 16. In this manner, the insertion tool 370 can be moved from its first configuration (FIG. 15) to its second configuration (FIG. 16). When the insertion tool 370 is in its second configuration, the distal end portion 372 of the inner shaft 371 can remain within the tool engagement opening 356 of the actuator 350 and the inner surface of the side wall 386 can be moved away from the side surface 320 of the screw 310. Thus, when the side surface 320 of the screw 310 is no longer disposed within the opening 387, the spacers 330A, 330B, 330C and 330D can be moved radially in an outward direction. Said another way, when the side surface 320 of the screw 310 is no longer disposed within the opening 387, the bone fixation device 300 can be moved from its first configuration to its second configuration.

As shown by the arrow KK in FIG. 16, the inner shaft 371 can be rotated relative to the outer shaft 381. When the inner shaft 371 is rotated, the actuator 350 of the bone fixation device 300 is rotated. More particularly, because the bone screw 310 is disposed within the bone tissue, rotation of the inner shaft 371 results in rotation of the actuator 350 relative to the bone screw 310. In this manner, the bone fixation device 300 can be moved from its first configuration (see e.g., FIGS. 5-7) to its second configuration (see e.g., FIGS. 8-10) using the same tool that is used to advance the bone screw 310 into the bone tissue.

FIGS. 17-20 show a bone fixation device 400 according to an embodiment. The bone fixation device 400 includes a bone screw 410, a locking screw 450 and a spacer 430. The bone screw 410 includes a proximal end portion 411 and a distal end portion 412, and defines a longitudinal axis A_{L}. The distal end portion 412 of the bone screw 410 includes a threaded portion 414 and a self-tapping tip 424. In this manner, the bone screw 410 can be advanced into a bone tissue, as described in more detail herein. As shown in FIG. 19, the bone screw 410 defines a lumen 413 therethrough. Similarly stated, the bone screw 410 is a cannulated screw that can be disposed about a guide member, such as, for example, a guide wire, a Kirschner wire (i.e., a K-wire) and/or the like.

As shown in FIG. 20, the proximal end portion 411 of the bone screw 410 defines an opening 418 that includes a threaded portion 419. Although the opening 418 is shown as being substantially coaxial with the longitudinal axis A_{L} and/or the lumen 413, in other embodiments the opening 418 can be offset from the longitudinal axis A_{L} and/or the lumen 413. At least a portion of the opening 418 is configured to engage a portion of an insertion and/or adjustment tool (not shown in FIGS. 17-20). More specifically, the proximal portion of the opening 418 is a hexagonally-shaped recess configured to receive a corresponding tip of the insertion tool. In this manner, the screw can be rotationally advanced (i.e., threaded) into a bone tissue via the insertion tool.

As described in more detail herein, the locking screw 450 is configured to limit the movement of the spacer 430 relative to the bone screw 410. The locking screw 450 has a proximal end portion 451 and a distal end portion 452, and defines a lumen 453 therethrough. The distal end portion 452 includes a threaded portion 454 that corresponds to (i.e., has substantially the same nominal size and thread pitch) the threaded portion 419 of the proximal end portion 411 of the bone screw 410. Similarly stated, the threaded portion 454 of the locking screw 450 includes male threads that correspond to the female threads within the opening 418 of the bone screw 410. Thus, the locking screw 450 can be threadedly coupled to the bone screw 410 such that that the lumen 453 of the locking screw 450 is substantially coaxial with the longitudinal axis A_{L}. In this arrangement, rotation of the locking screw 450 relative to the bone screw 410 results in movement of the locking screw 450 relative to the bone screw 410 along the longitudinal axis A_{L}.

The proximal end portion 451 of the locking screw 450 includes a tool engagement opening 456. The tool engagement opening 456 is configured to receive and/or engage a portion of an insertion and/or adjustment tool (not shown in FIGS. 17-20). More specifically, the tool engagement opening 456 is a hexagonally-shaped recess configured to receive a corresponding tip of the insertion tool. Thus, as described in more detail below, an insertion tool can be used to move the locking screw 450 relative to the bone screw 410 (e.g., to tighten the locking screw 450 onto the proximal end portion 411 of the bone screw 410).

The spacer 430 includes a distal end surface 431, a proximal end surface 434, and a side surface 436. The spacer 430 defines a first lumen 437 and a second lumen 438. As shown in FIG. 19, the side wall of the spacer 430 defining the first lumen 437 includes a shoulder 439. Similarly stated, the first lumen 437 includes a counter-bored opening defined by the proximal end surface 434 and a counter-bored opening defined by the distal end surface 431. In this manner, the spacer 430 can be disposed between the bone screw 410 and the locking screw 450 such that the shoulder 439 is in contact with the proximal end portion 451 (i.e., the head) of the locking screw 450 and the proximal end portion 411 of the bone screw 410. Similarly stated, the spacer 430 can be disposed between the bone screw 410 and the locking screw 450 such that the proximal end portion 451 of the locking screw 450 is disposed within the counter-bored opening defined by the proximal end surface 434 of the spacer 430 and the proximal end portion 411 of the bone screw 410 is disposed within the counter-bored opening defined by the distal end surface 431 of the spacer 430. Thus, when the spacer 430 is disposed between the bone screw 410 and the locking screw 450 and when the locking screw 450 is threaded into the proximal end portion 411 of the bone screw 410, the locking screw 450 and the bone screw 410 exert a compressive force on the shoulder 439 of the spacer 430. In this manner, when the locking screw 450 is tightened onto the bone screw 410, the spacer 430 can be retained on the bone screw 410. Moreover, when the locking screw 450 is tightened onto the bone screw 410, rotational and/or longitudinal movement of the spacer 430 relative to the bone screw can be limited.

The second lumen 438 of the spacer 430 is configured to receive a portion of an insertion and/or actuation tool, such as, for example, the actuation tool 470 shown in FIG. 21. In this manner, the spacer 430 can be rotated about the longitudinal axis A_{L} of the bone screw 410 using the actuation tool 470, as shown by the arrow LL in FIG. 17 and as described in more detail below. In some embodiments, the side wall of the spacer 430 defining the second lumen 438 can be configured to limit the axial movement of the spacer 430 with respect to the insertion and/or actuation tool. For example, in some embodiments, the side surface defining the second lumen 438 can define openings and/or grooves configured to receive a snap ring, clip, E-ring or any other suitable mechanism for removably coupling the spacer 430 to the insertion and/or actuation tool.

The side surface 436 can have any suitable shape and/or contour configured to contact and/or engage a portion of a bone structure. In some embodiments, the side surface 436 can have a shape that corresponds to a shape of a bone structure. For example, in some embodiments, a portion of the side surface 436 can be concave such that the side surface 436 forms a saddle to receive a portion of the bone structure. Although the side surface 436 is shown as being asymmetrical about the longitudinal axis of the first lumen 437, in other embodiments the side surface 436 can be substantially symmetrical about the longitudinal axis of the first lumen 437. Similarly stated, although the side surface 436 is shown as being a cam surface, in other embodiments, the side surface 436 need not have a cam lobe and/or cam profile.

The distal surface 431 of the spacer 430 includes a set of protrusions 440. The protrusions 440 each includes a sharpened tip such that when the spacer 430 is disposed against a bone tissue, the protrusions can advance into the bone tissue, as described in more detail herein.

The bone fixation device 400 can be inserted into a body (not shown) using the actuation tool 470 shown in FIG. 21. The actuation tool 470 includes an outer shaft 481 and an inner shaft 471 disposed within the outer shaft 481. The outer shaft 481 defines a lumen 485 therethrough and a longitudinal axis A_{L}. A distal end portion 482 of the outer shaft 481 includes a side wall 486 that defines an opening 487 and a protrusion 488. The opening 487 is configured to receive a portion of the spacer 430. More specifically, the inner surface of the side wall 486 has a shape corresponding to at least a portion of a side surface of the spacer 430. The protrusion 488 is configured to be disposed within the second lumen 438 of the spacer. In this manner, when the spacer 430 is disposed within the opening 487, rotation of the outer shaft 481 of the actuation tool 470 results in rotation of the spacer 430 relative to the bone screw 410 about the longitudinal axis A_{L}, as shown by the arrow MM in FIG. 21. Thus, when the bone fixation device 400 is disposed within the body, the orientation of the spacer 430 relative to the bone screw 410 can be adjusted using the actuation tool 470.

The inner shaft 471 of the actuation tool 470, which can also be referred to as the hex driver shaft, is movably disposed within the lumen 485 of the outer shaft 481. In this manner, the inner shaft 471 can be rotated relative to the outer shaft 481, as shown by the arrow NN. The inner shaft 471 of the actuation tool 470 defines a lumen 475 that is coaxial with the longitudinal axis A_{L}. The lumen 475 is configured to be aligned with the lumen 413 of the bone screw 410 when the bone fixation device 400 is engaged with the actuation tool 470. In this manner, a guide wire, a Kirschner wire or the like can be disposed through the lumen 475 of the inner shaft 471 and the lumen 413 of the screw 410 to facilitate the insertion of the bone fixation device 400 into a bone tissue.

A distal end portion 472 of the inner shaft 471 includes a set of hexagonal-shaped surfaces corresponding to the hexagonal-shaped tool engagement opening 456 defined by the locking screw 450. In this manner, the distal end portion 472 of the inner shaft 471 can be received within the tool engagement opening 456 of the locking screw 450 such that rotation of the inner shaft 471 about the longitudinal axis A_{L} results in rotation of the actuator 450. Moreover, the inner shaft 471 can be rotated relative to the outer shaft 481 such that the locking screw 450 can be tightened onto the bone screw 410 while the spacer 430 is maintained in a fixed position via the outer shaft 481 of the actuation tool 470.

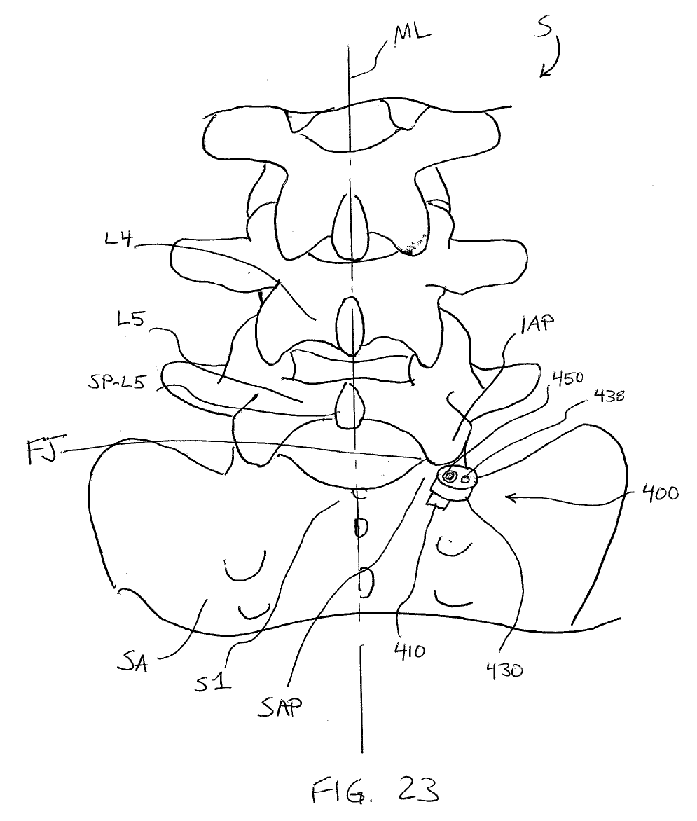

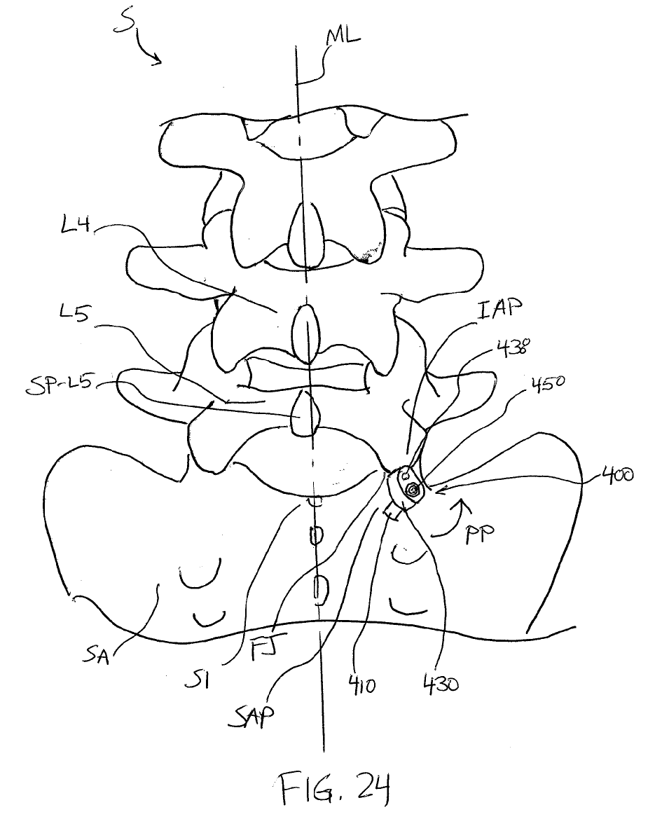

The bone fixation devices device described herein can be inserted and deployed within the body to stabilize and/or fix the L5-S1 location of the spinal column. FIG. 22 is a flow chart of a method 500 for disposing a bone fixation device within a body, according to an embodiment. The method illustrated in FIG. 22 is discussed with reference to FIGS. 23 and 24, which are perspective views of the bone fixation device 400 (as discussed with reference to FIGS. 17-20) disposed within a portion of a spine S in a first configuration (FIG. 23) and a second configuration (FIG. 24). The spine S has a midline ML axis, a sacrum SA, a fifth lumbar vertebra L5, and a first sacral vertebra S1. The fifth lumbar vertebra L5 includes a spinous process SP-L5 and an inferior articulate process IAP. The first sacral vertebra includes a superior articulate process SAP. A region between the inferior articulate process IAP and the superior articulate process SAP defines a facet joint FJ. Although the method 500 is discussed with reference to the bone fixation device 400, the method 500 can be performed with any suitable bone fixation device such as the types shown and described herein (e.g., bone fixation device 300). Similarly, although the method 500 is discussed with reference to disposing a bone fixation device in a particular bone structure and/or in a particular orientation, in other embodiments, the method 500 can include disposing a bone fixation device in any suitable bone structure and/or in any suitable orientation.

The method 500 includes inserting a bone stabilizer into a body, at 502. The bone stabilizer, which can be any bone fixation device such as the types shown and described herein, includes a bone screw, a spacer coupled to a proximal end portion of the bone screw, and a retention screw. The bone stabilizer can be inserted in any suitable manner. For example, in some embodiments, the bone stabilizer can be inserted into the body percutaneously and/or in a minimally-invasive manner. In some embodiments, the bone stabilizer can be inserted through a lateral skin incision (i.e., a skin incision offset from the midline axis ML of the spine S). The lateral skin incision can have a length of between 3 mm and 25 mm. In some embodiments, for example, the lateral skin incision can have a length of approximately 10 mm. Moreover, in some embodiments, the bone stabilizer can be inserted into the body via a cannula (not shown in FIGS. 23-24). In some embodiments, such a cannula can have a size of between 3 mm and 25 mm. In some embodiments, for example, the size of the cannula can be approximately 10 mm.

The bone stabilizer can be inserted in a single operation or in multiple operations. For example, in some embodiments, a bone stabilizer (e.g., the bone fixation device 300) can be coupled to an insertion tool (e.g., insertion tool 370 shown and described above with reference to FIGS. 14-16) and inserted into the body in a single operation. In other embodiments, a bone stabilizer (e.g., the bone fixation device 400) can be inserted into the body in several discrete operations. For example, referring to FIG. 23, in some embodiments, the bone screw 410 of the bone fixation device 400 can be inserted into the body and advanced into a superior articulate process SAP of the sacrum SA. The spacer 430 and the locking screw 450 can then be disposed about the proximal end portion 411 of the bone screw 410.

Returning to the flow chart shown in FIG. 22, the spacer is moved relative to the bone screw from a first position to a second position using an actuation tool, at 504. In some embodiments, the spacer can be moved using the same tool used to insert the spacer into the body and/or advance the spacer into the bone tissue. For example, in some embodiments, the spacer can be the spacer 330A, and can be moved relative to the screw 310 by rotating the actuator 350 using the tool 370, as shown and described above. In other embodiments, the spacer can be the spacer 430, and can be rotated relative to the bone screw 410 via the actuation tool 470, as shown and described above, and as shown by the arrow PP in FIG. 24.

In some embodiments, the spacer can be moved such that a portion of the spacer contacts and/or moves a portion of a bone tissue. Referring to FIG. 24, in some embodiments, the spacer 430 can be rotated relative to the bone screw 410 such that a side surface 436 of the spacer 430 contacts the inferior articulate process IAP of the L5 vertebra. In some embodiments, the movement of the spacer 430 can cause the inferior articulate process IAP and/or a portion of the L5 vertebra to be moved in a cephalic direction. Similarly stated, in some embodiments, the movement of the spacer 430 can distract a portion of the L5 vertebra relative to the S1 vertebra. In this manner, the spacer 430 can stabilize and/or fix a portion of the spine.

Returning to the flow chart shown in FIG. 22, the retention screw is rotated relative to the bone screw using the actuation tool such that the spacer is maintained in the second position, at 506. In this manner, the same tool used to move the spacer can be used to tighten the retention screw, thereby preventing the spacer from rotating relative to the bone screw. In some embodiments, the retention screw can be the locking screw 450 of the bone fixation device 400 and can be rotated using the actuation tool 470 shown and described above. In addition to limiting the rotation of the spacer 430 relative to the bone screw 410, when the locking screw 450 is tightened, the locking screw 450 exerts a force in the distal direction on the spacer 430. In this manner, the protrusions 440 of the spacer 430 can be inserted into the superior articulate process SAP of the sacrum SA, thereby providing additional fixation of the spacer 430 to the sacrum SA.

When the spacer 430 is locked into position, the bone fixation device 400 can limit the extension of spinal column while allowing flexion of the spinal column in the L5-S1 region. Similarly stated, when the spacer 430 is locked into position, the bone fixation device 400 dynamically stabilizes a portion of the spinal column S.

Although various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Thus, the breadth and scope of the invention should not be limited by any of the above-described embodiments. While the invention has been particularly shown and described with reference to specific embodiments thereof, it will be understood that various changes in form and details may be made.

For example, although the spacers 330A, 330B, 330C and 330D are shown and described above as being matingly coupled to the screw 310 via the protrusions 322A, 322B, 322C and 322D and grooves 332A, 332B, 332C and 332D, in other embodiments, a spacer can be matingly and/or movably coupled to a screw and/or an actuator using any suitable mechanism. In some embodiments, for example, a spacer can be matingly coupled to a screw and/or an actuator by a dove tail fitting such that movement of the spacer relative to the screw and/or actuator is limited. More particularly, in some embodiments, an actuator and/or screw can define a groove having a trapezoidal cross-sectional shape. A spacer can define a protrusion having a trapezoidal shape that corresponds to the shape of the groove. The protrusion of the spacer can be disposed within the groove of the screw and/or actuator. In this manner, the spacer can be maintained in sliding contact with the screw and/or the actuator.

Although the actuators shown and described above (e.g., actuator 350) include an angled surface corresponding to an angled surface of a spacer, in other embodiments, the actuator need not include an angled surface. For example, in some embodiments, a portion of a screw can include an angled surface corresponding to an angled surface of a spacer.

Although the second lumen 438 of the spacer 430 is shown as being distinct from the first lumen 437, in other embodiments, the second lumen 438 can share a common boundary with the first lumen 437. Similarly stated, although the second lumen 438 is shown as being non-contiguous with the first lumen 437, in other embodiments, the second lumen 438 can be contiguous with the first lumen 437.

Although the second lumen 438 of the spacer 430 is shown as being substantially parallel to the first lumen 437, in other embodiments, the second lumen 438 can be non-parallel to the first lumen 437. Similarly stated, although a longitudinal axis of the second lumen 438 is shown as being substantially parallel to and offset from a longitudinal axis of the first lumen 437, in other embodiments, a longitudinal axis of the second lumen 438 can intersect a longitudinal axis of the first lumen 437.

Although the second lumen 438 of the spacer 430 is shown as extending through the spacer, in other embodiments, the second lumen 438 can be a blind hole.

Although various embodiments have been described as having particular features and/or combinations of components, other embodiments are possible having a combination of any features and/or components from any of embodiments as discussed above. For example, in some embodiments, a bone fixation device can include a primary spacer that is rotatable relative to a bone screw, such as spacer 430 and a set of secondary spacers that can be moved radially relative to the bone screw, such as spacer 330A, 330B, 330C and 330D. In other embodiments, a bone fixation device can include multiple spacers, such as spacer 330A, 330B, 330C and 330D in series (i.e., longitudinally disposed and in contact with an adjacent spacer).

Furthermore, any of the various embodiments and applications of method 500 may employ any of the various embodiments of the bone fixation devices disclosed herein.

What is claimed is:

1. An apparatus, comprising:
a screw having a distal end portion and a proximal end portion, the distal end portion configured to be threaded into a bone tissue, the proximal end portion having a surface;
an actuator threadedly coupled to the screw, the actuator having an actuation surface defining a line substantially non-parallel to and substantially non-normal to a longitudinal axis of the screw; and
a spacer having a first surface substantially parallel to and in contact with the surface of the proximal end portion of the screw and a second surface substantially parallel to and in contact with the actuation surface of the actuator,
the actuator configured to move the spacer relative to the screw between a first position and a second position.

2. The apparatus of claim 1, wherein the actuator is configured to move relative to the screw through a range of motion when moving the spacer between the first position and the second position, the line of the actuation surface being substantially non-parallel to and substantially non-normal to the longitudinal axis of the screw throughout the range of motion.

3. The apparatus of claim 1, wherein a third surface of the spacer is spaced apart from the longitudinal axis of the screw by a first distance when the spacer is in the first position, the third surface of the spacer is spaced apart from the longitudinal axis of the screw by a second distance when the spacer is in the second position, the second distance greater than the first distance.

4. The apparatus of claim 1, wherein the spacer is configured to move radially relative to the screw when moved between the first position and the second position.

5. The apparatus of claim 1, wherein a shape of the spacer when the spacer is in the first position is substantially the same as a shape of the spacer when the spacer is in the second position.

6. The apparatus of claim 1, further comprising:
a second spacer having a first surface substantially parallel to and in contact with the surface of the screw and a second surface substantially parallel to and in contact with the actuation surface of the actuator, the actuator configured to move the second spacer relative to the screw between a first position and a second position,
the first spacer and the second spacer collectively having a first outer diameter when the first spacer is in its first position and the second spacer is in its first position, the first outer diameter less than a maximum outer diameter of the screw, the first spacer and the second spacer collectively having a second outer diameter when the first spacer is in its second position and the second spacer is in its second position, the second outer diameter greater than the maximum outer diameter of the screw.

7. The apparatus of claim A6, wherein the second outer diameter is greater than the first outer diameter by between 1 millimeter and 2 millimeters.

8. The apparatus of claim 1, wherein the spacer is matingly and slidably coupled to at least one of the surface of the proximal end portion of the screw or the actuation surface of the actuator.

9. The apparatus of claim 1, wherein:
the first surface of the spacer defines a groove; and
the surface of the proximal end portion of the screw includes a protrusion configured to be matingly received within the groove such that the first surface of the spacer is matingly and movably coupled to the surface of the proximal end portion of the screw.

10. The apparatus of claim 1, wherein:
the actuator is configured to move relative to the screw along the longitudinal axis of the screw when the actuator moves the spacer between the first position and the second position; and
the spacer is configured to move radially relative to the screw when moved between the first position and the second position.

11. The apparatus of claim 1, wherein:
the proximal end portion of the screw defines a threaded opening; and
the distal end portion of the actuator is threadedly disposed within the opening of the proximal end portion of the screw,
the actuator is configured to rotate relative to the screw when the actuator moves the spacer between the first position and the second position.

12. An apparatus, comprising:
a spine stabilizer having a proximal end portion and a distal end portion, the distal end portion configured to be threaded into a bone tissue, the proximal end portion including a spacer having a bone engagement surface,
the bone engagement surface having a first shape and being spaced apart from a longitudinal axis of the spine stabilizer by a first distance when the spine stabilizer is in a first configuration, the bone engagement surface having a second shape and being spaced apart from the longitudinal axis of the spine stabilizer by a second distance when the spine stabilizer is in a second configuration, the second distance greater than the first distance, the second shape substantially the same as the first shape.

13. The apparatus of claim 12, wherein:
the proximal end portion of the spine stabilizer has a tool engagement portion configured to engage an insertion tool such that at least a portion of the spine stabilizer is rotatable by the insertion tool to thread the distal end portion of the spine stabilizer into the bone tissue;
the spacer is a first spacer; and
the proximal end portion of the spine stabilizer includes a second spacer, the first spacer and the second spacer collectively having a first outer diameter when the spine stabilizer is in the first configuration, the first outer diameter less than a maximum outer diameter of the tool engagement portion, the first spacer and the second spacer collectively having a second outer diameter when the spine stabilizer is in the second configuration, the second outer diameter greater than the maximum outer diameter of the tool engagement portion.

14. The apparatus of claim 12, wherein the spacer is configured to move radially relative to the longitudinal axis when the spine stabilizer is moved between the first configuration and the second configuration.

15. The apparatus of claim 12, wherein the spine stabilizer has an actuator configured to move the spine stabilizer between the first configuration and the second configuration, the actuator having an actuation surface, a line defined by the actuation surface being non-parallel to and non-normal to the longitudinal axis,
the spacer having a surface substantially parallel to and in contact with the actuation surface of the actuator.

16. The apparatus of claim 12, wherein the spacer is matingly and slidably coupled to the proximal end portion of the spine stabilizer.

17. The apparatus of claim 12, wherein:
a surface of the spacer defines a groove; and
a surface of the proximal end portion of the spine stabilizer includes a protrusion configured to be matingly received within the groove such that the surface of the spacer is matingly and movably coupled to the surface of the spine stabilizer.

18. An apparatus, comprising:
a bone screw having a distal end portion and a proximal end portion, the distal end portion configured to be threaded into a bone tissue; and
a spacer movably coupled to the proximal end portion of the bone screw, the spacer having a bone engagement surface, the spacer defining a first opening and a second opening, the first opening configured to receive at least a portion of the proximal end portion of the bone screw, the second opening configured to receive a portion of an insertion tool.

19. The apparatus of claim 18, wherein the second opening is distinct from the first opening.

20. The apparatus of claim 18, wherein the bone engagement surface of the spacer is asymmetrical about a longitudinal axis of the bone screw.

21. The apparatus of claim 18, wherein a longitudinal axis of the first opening is substantially parallel to a longitudinal axis of the second opening.

22. The apparatus of claim 18, further comprising a retention screw having a portion configured to be disposed within the first opening and threadedly coupled to the proximal end portion of the bone screw, the retention screw configured to limit movement of the spacer relative to the bone screw.

23. A method, comprising:
inserting a bone stabilizer into a body, the bone stabilizer including a bone screw, a spacer coupled to a proximal end portion of the bone screw, and a retention screw;
moving the spacer relative to the bone screw from a first position to a second position using an actuation tool; and
rotating the retention screw relative to the bone screw using the actuation tool such that the spacer is maintained in the second position.

24. The method of claim 23, wherein the inserting is performed using the actuation tool.

25. The method of claim 23, wherein:
the inserting includes threading the bone screw into a pedicle of an S1 vertebra; and
when the spacer is in the second position, a bone engagement surface of the spacer is configured to contact a portion of an inferior articulate process of an L5 vertebra.
